(19) United States
(12) Patent Application Publication
(10) Pub. No.: US 2011/0144382 A1
(43) Pub. Date: Jun. 16, 2011

(54) ADVANCED CATALYSTS FOR FINE CHEMICAL AND PHARMACEUTICAL APPLICATIONS

(75) Inventors: Qinghua Yin, Tempe, AZ (US); Xiwang Qi, Scottsdale, AZ (US)

(73) Assignee: SDCMATERIALS, INC., Tempe, AZ (US)

(21) Appl. No.: 12/969,264

(22) Filed: Dec. 15, 2010

Related U.S. Application Data
(60) Provisional application No. 61/284,329, filed on Dec. 15, 2009.

(51) Int. Cl. B01J 23/42; B01J 23/00; B01J 23/46; B01J 21/02; B01J 37/00; C07C 201/12; C07C 209/36; C07C 67/00; B82Y 30/00; B82Y 40/00

(52) U.S. Cl. 562/400; 502/100; 502/339; 502/300; 502/355; 502/334; 568/939; 564/417; 568/300; 977/773; 977/840

(57) ABSTRACT

A catalyst comprising a plurality of support nanoparticles and a plurality of catalytic nanoparticles. At least one catalytic nanoparticle is bonded to each support nanoparticle. The catalytic particles have a size and a concentration, wherein a first configuration of the size and the concentration of the catalytic nanoparticles enables a first catalysis result and a second configuration of the size and the concentration of the catalytic nanoparticles enables a second catalysis result, with the first and second configurations having a different size or concentration, and the first and second catalysis results being different. In some embodiments, the first catalysis result is a selective reduction of a first selected functional group without reducing one or more other functional groups, and the second catalysis result is a selective reduction of a second selected functional group without reducing one or more other functional groups.

### CROSS-REFERENCE TO RELATED APPLICATIONS

This application claims priority to U.S. Provisional Patent Application Ser. No. 61/284,329, filed Dec. 15, 2009 and entitled “MATERIALS PROCESSING,” which is hereby incorporated herein by reference in its entirety as if set forth herein.

### FIELD OF THE INVENTION

The present invention relates to the field of catalysts. More specifically, the present invention relates to catalysts that are finely-tuned to a particular size and a particular concentration to achieve a particular catalysis result.

### BACKGROUND OF THE INVENTION

Hydrogenation is a chemical reaction that results in an addition of hydrogen (H₂) to a molecule. Wilkinson's catalyst, a common name for tris-(triphenylphosphine)rhodium chloride, is used to hydrogenate some functional groups of the molecule without affecting others. Wilkinson's catalyst is a homogeneous catalyst based on rhodium (Rh), having the formula RhCl(PPh₃)₃. Wilkinson's catalyst can reduce, for example, an olefin functional group over a nitro functional group, hydrogenating the double bond of the molecule, but leaving the NO₂ functional group intact.

However, Wilkinson's catalyst suffers from a number of shortcomings. First, Rh ions are very expensive. Additionally, it is difficult to remove Rh ions from the catalyst afterwards because the catalyst is homogeneous. Furthermore, a purification system to remove the Rh ions is complex and costly, as the silica that is used to remove the Rh ions is expensive.

### SUMMARY OF THE INVENTION

The present invention provides catalysts that are finely-tuned to achieve particular catalysis results, such as the selective reduction of a selected functional group without reducing one or more other functional groups of a molecule. The size and concentration of the catalytic particles in the catalyst are configured to achieve a particular catalysis result, but can be adjusted to achieve a different catalysis result even though the catalyst still consists of the same chemical elements.

In one aspect of the present invention, a catalyst comprises a plurality of support nanoparticles and a plurality of catalytic nanoparticles. At least one catalytic nanoparticle is bonded to each support nanoparticle. The catalytic particles have a size and a concentration, wherein a first configuration of the size and the concentration of the catalytic nanoparticles enables a first catalysis result and a second configuration of the size and the concentration of the catalytic nanoparticles enables a second catalysis result, with the first and second configurations having a different size or concentration, and the first and second catalysis results being different.

In some embodiments, the first catalysis result is a selective reduction of a first selected functional group without reducing one or more other functional groups of a molecule, and the second catalysis result is a selective reduction of a second selected functional group without reducing one or more other functional groups of a molecule, wherein the first and second selected functional groups are different. In some embodiments, the first selected functional group is olefin. In some embodiments, the first selected functional group is nitro. In some embodiments, the first selected functional group is ketone. In some embodiments, the first catalysis result is a selective reduction of the olefin functional group without reducing the nitro functional group of a molecule, and the second catalysis result is a selective reduction of the nitro functional group without reducing the halide functional group of a molecule. In some embodiments, the first catalysis result is a selective reduction of the olefin functional group without reducing the nitro functional group of a molecule, and the second catalysis result is a selective reduction of the ketone functional group without reducing the ester functional group of a molecule. In some embodiments, the first catalysis result is a selective reduction of the nitro functional group without reducing the halide functional group of a molecule, and the second catalysis result is a selective reduction of the ketone functional group without reducing the ester functional group of a molecule.

In some embodiments, the catalytic nanoparticles are platinum nanoparticles. In some embodiments, the catalytic nanoparticles are alloy nanoparticles. In some embodiments, the alloy nanoparticles comprise platinum and rhodium. In some embodiments, the support nanoparticles are aluminum oxide nanoparticles. In some embodiments, the catalytic nanoparticles are platinum nanoparticles. In some embodiments, the catalytic nanoparticles are platinum-rhodium nanoparticles.

In another aspect of the present invention, a method of forming a catalyst comprises: determining a first particular configuration for a first catalyst, wherein the first particular configuration comprises a particular size and concentration of catalytic nanoparticles configured to achieve a first particular catalysis result when the first catalyst is used in a catalytic process; and forming the first catalyst according to the first particular configuration, wherein the first catalyst comprises a plurality of support nanoparticles each having at least one catalytic nanoparticle bonded to it.

In some embodiments, the first particular catalysis result is a selective reduction of a selected functional group without reducing one or more other functional groups of a molecule. In some embodiments, the first selected functional group is olefin. In some embodiments, the first selected functional group is nitro. In some embodiments, the first selected functional group is ketone. In some embodiments, the first particular catalysis result is a selective reduction of the olefin functional group without reducing the nitro functional group of a molecule. In some embodiments, the first particular catalysis result is a selective reduction of the nitro functional group without reducing the halide functional group of a molecule. In some embodiments, the first particular catalysis result is a selective reduction of the ketone functional group without reducing the ester functional group of a molecule.

In some embodiments, the catalytic nanoparticles are platinum nanoparticles. In some embodiments, the catalytic nanoparticles are alloy nanoparticles. In some embodiments, the alloy nanoparticles comprise platinum and rhodium. In some embodiments, the support nanoparticles are aluminum oxide nanoparticles. In some embodiments, the catalytic nanoparticles are platinum nanoparticles. In some embodiments, the catalytic nanoparticles are platinum-rhodium nanoparticles.

In some embodiments, the step of forming the first catalyst comprises: vaporizing support material and catalytic material using a plasma gun, thereby forming vaporized support material and vaporized catalytic material; and quenching the vaporized support material and the vaporized catalytic material, thereby forming the support nanoparticles and the catalytic nanoparticles.

In some embodiments, the method further comprises: determining a second particular configuration for a second catalyst, wherein the second particular configuration comprises a particular size and concentration of catalytic nanoparticles configured to achieve a second particular catalysis result when the second catalyst is used in a catalytic process; and forming the second catalyst according to the second particular configuration, wherein the second catalyst comprises a plurality of support nanoparticles each having at least one catalytic nanoparticle bonded to it. The second catalyst comprises the same chemical elements as the first catalyst, but the second particular configuration differs from the first particular configuration in at least the size or the concentration of catalytic nanoparticles, thereby enabling the second particular catalysis result to be different from the first particular catalysis result.

In yet another aspect of the present invention, a method of using a catalyst comprises: providing a finely-tuned catalyst, wherein the finely-tuned catalyst comprises a plurality of support nanoparticles and a plurality of catalytic nanoparticles, with at least one catalytic nanoparticle being bonded to each support nanoparticle; and using the finely-tuned catalyst in a catalytic process, wherein the finely-tuned catalyst enables selective reduction of a selected functional group without reduction of one or more other functional groups of a molecule.

In some embodiments, the selected functional group is olefin. In some embodiments, the finely-tuned catalyst enables selective reduction of the olefin functional group without reduction of the nitro functional group.

In some embodiments, the selected functional group is nitro. In some embodiments, the finely-tuned catalyst enables selective reduction of the nitro functional group without reduction of the halide functional group.

In some embodiments, the selected functional group is ketone. In some embodiments, the finely-tuned catalyst enables selective reduction of the ketone functional group without reduction of the ester functional group.

In some embodiments, the catalytic nanoparticles are platinum nanoparticles. In some embodiments, the catalytic nanoparticles are alloy nanoparticles. In some embodiments, the alloy nanoparticles comprise platinum and rhodium. In some embodiments, the support nanoparticles are aluminum oxide nanoparticles. In some embodiments, the catalytic nanoparticles are platinum nanoparticles. In some embodiments, the catalytic nanoparticles are platinum-rhodium nanoparticles.

### BRIEF DESCRIPTION OF THE DRAWINGS

FIG. 1 illustrates one embodiment of a catalytic process that involves reducing the olefin functional group over the nitro functional group in accordance with the principles of the present invention.

FIG. 2 illustrates one embodiment of a catalytic process that involves reducing the nitro functional group over the halide functional group in accordance with the principles of the present invention.

FIG. 3 illustrates one embodiment of a catalytic process that involves non-selective hydrogenation in accordance with the principles of the present invention.

FIGS. 4a-c illustrate one embodiment of a catalytic process that involves deprotection in accordance with the principles of the present invention.

FIG. 5 illustrates one embodiment of a method of forming and using catalysts in accordance with the principles of the present invention.

### DETAILED DESCRIPTION OF THE INVENTION

The following description is presented to enable one of ordinary skill in the art to make and use the invention and is provided in the context of a patent application and its requirements. Various modifications to the described embodiments will be readily apparent to those skilled in the art and the generic principles herein may be applied to other embodiments. Thus, the present invention is not intended to be limited to the embodiment shown but is to be accorded the widest scope consistent with the principles and features described herein.

Various aspects of the disclosure may be described through the use of flowcharts. Often, a single instance of an aspect of the present disclosure may be shown. As is appreciated by those of ordinary skill in the art, however, the protocols, processes, and procedures described herein may be repeated continuously or as often as necessary to satisfy the needs described herein. Additionally, it is contemplated that certain method steps of the invention can be performed in alternative sequences to those disclosed in the flowcharts. Accordingly, the scope of the claims should not be limited to any specific order of method steps unless the order is required by the language of the claims.

Embodiments of the present invention are directed to advanced catalysts, and are particularly useful for fine chemicals and pharmaceutical applications. In some embodiments, the advanced catalysts are used in continuous reactions. In a continuous reaction, materials pass through a tube to create a reaction. The continuous reaction can be a selective hydrogenation reaction (where one functional group is hydrogenated over one or more other functional groups), a non-selective hydrogenation reaction (where every functional group has an equal chance of being hydrogenated), an oxidation reaction, or an isomerization reaction. It is contemplated that other continuous reactions are also within the scope of the present invention. Alternatively, the advanced catalysts can be used in batch reactions. In a batch reaction, you have a container or vessel, such as a pot. The catalyst is placed in the container or vessel along with the material that you want to react to the catalyst. The batch reaction can be a hydrogenation reaction (selective or non-selective), an oxidation reaction, a carbon-carbon bond formation reaction, or an isomerization reaction. It is contemplated that other batch reactions are also within the scope of the present invention.

As discussed above, hydrogenation is a chemical reaction that results in an addition of hydrogen (H₂) to a molecule. In some embodiments, the hydrogenation reaction is nonselective. A nonselective hydrogenation reaction takes only one path. A catalyst is used to hydrogenate functional groups of a molecule non-selectively. Here, every functional group of the molecule has an equal chance of being hydrogenated or reduced. FIG. 3 illustrates one embodiment of a catalytic process that involves non-selective hydrogenation in accordance with the principles of the present invention, where each hydrogen functional group of benzene is non-selectively hydrogenated, thereby producing cyclohexane.

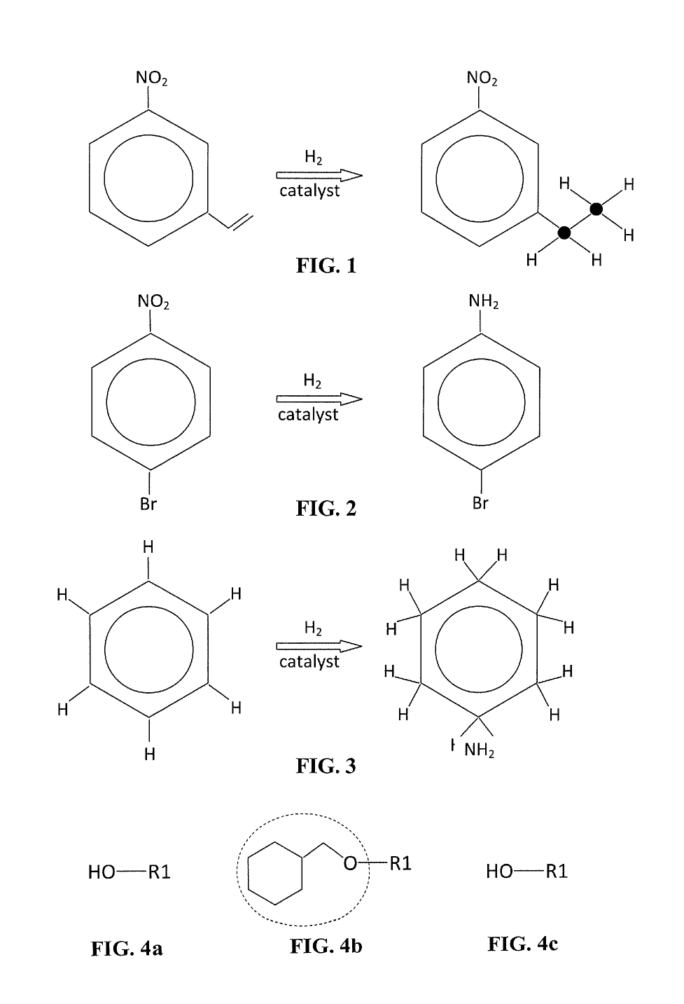

In a selective hydrogenation reaction, a catalyst is used to hydrogenate one or more functional groups of a molecule without affecting others. The advanced catalysts of the present invention are able to perform selective hydrogenation in batch reactions. For example, the advanced catalysts are able to selectively hydrogenate an olefin functional group over a nitro functional group (such as illustrated in FIG. 1), a nitro functional group over a halide functional group (such as illustrated in FIG. 2), and a ketone functional group over an ester functional group.

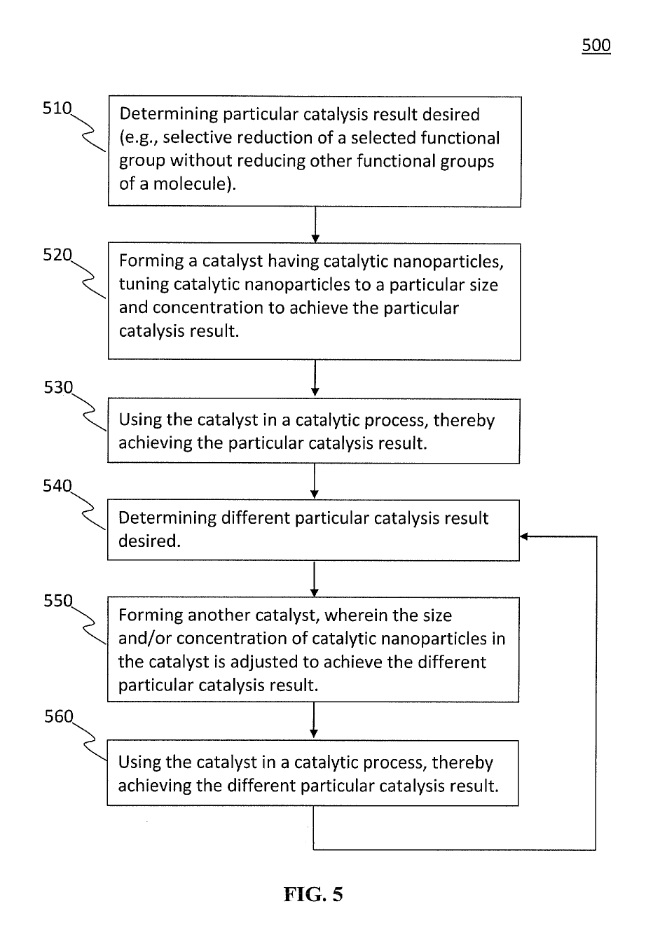

The nanoparticles of the present invention are also able to be used in conjunction with deprotection. A protecting group is introduced into a compound by modifying a functional group in order to obtain chemoselectivity in a subsequent reaction. FIGS. 4a-c illustrate a deprotection process. Looking at FIG. 4a, say that you want the oxygen to be attached to R1, but you do not want the oxygen to react to anything else. In FIG. 4b, you can attach a benzyl group to the oxygen in order to preserve the oxygen presence there. The benzyl group acts as a protecting group, represented by the dotted boundary. You can then react whatever else you want on R1. Then, you remove the benzyl group, as seen in FIG. 4c, and you can react the oxygen with whatever functional group you want.

The advanced catalysts of the present invention preferably comprise nanoparticles. The nanoparticles of the present invention can be formed in a variety of ways. Formation methods and systems that have been found to be particularly useful and effective are described in the following U.S. Patent Applications, which are all hereby incorporated by reference as if set forth herein: U.S. patent application Ser. No. 12/001,643, filed Dec. 11, 2007, entitled “METHOD AND SYSTEM FOR FORMING PLUG AND PLAY METAL CATALYSTS”; U.S. patent application Ser. No. 12/474,081, filed May 28, 2009, entitled “METHOD AND SYSTEM FOR FORMING PLUG AND PLAY METAL CATALYSTS”; U.S. patent application Ser. No. 12/001,602, filed Dec. 11, 2007, entitled “METHOD AND SYSTEM FOR FORMING PLUG AND PLAY METAL COMPOUND CATALYSTS”; U.S. patent application Ser. No. 12/001,644, filed Dec. 11, 2007, entitled “METHOD AND SYSTEM FOR FORMING PLUG AND PLAY OXIDE CATALYSTS”; and U.S. Provisional Patent Appl. No. 61/284,329, filed Dec. 15, 2009, entitled “MATERIALS PROCESSING.” It is contemplated that any of these described methods and systems can be used to form the nanoparticles and catalysts of the present invention. Additionally, it is contemplated that other methods and systems can be employed within the scope of the present invention.

Each of the nanoparticles comprises nano-active material (e.g., catalytic nanoparticles) on nano-support (e.g., support or carrier nanoparticles). Size of the nano-active material on the nano-support has been observed to be a function of an initial ratio of active material and carrier material used in a processing chamber. It has been observed that as an amount of the active material increases in relation to the carrier material, the size of the resulting nano-active material increases. Likewise, as the amount of active material decreases in relation to the carrier material, the size of the resulting nano-active material decreases. In some embodiments, the advanced catalysts require the nano-active material to be of a specific size and the loading to be of a specific value by using a method as described under the section entitled “TUNABLE SIZE OF NANO-ACTIVE MATERIAL ON NANO-SUPPORT” in U.S. Provisional Patent Appl. No. 61/284,329, filed Dec. 15, 2009, entitled “MATERIALS PROCESSING.” In some embodiments, the advanced catalysts have specific concentration values of the nanoparticles.

In some embodiments, the advanced catalysts of the present invention are heterogeneous catalysts. Unlike in a homogeneous catalyst, the nanoparticles of the present invention's heterogeneous catalysts are suspended in a solvent. Since the advanced catalysts of the present invention are heterogeneous catalysts, a filtration system can be used for each reaction, which is simpler than a purification system for the homogeneous catalysts, such as Wilkinson's catalyst. In some embodiments, a piece of filter paper is used to filter out the nanoparticles that are suspended in the solvent.

As discussed above, the advanced catalysts of the present invention can be adjusted and tuned to achieve a particular catalysis result, such as selective hydrogenation. In some embodiments, the catalysts of the present invention is configured and used to selectively hydrogenate or reduce the olefin functional group over the nitro functional group. A configuration that has been found to be particularly effective in this catalysis result, as well as other catalysis results (e.g., selectively reducing a nitro functional group over a halide functional group, or a ketone functional group over an ester functional group) is a plurality of nanoparticles, with each nanoparticle comprising nano-active material on a nano-support. In some embodiments, the nano-support is alumina. In some embodiments, the nano-active material is platinum. In some embodiments, the nano-active material is an alloy of rhodium and platinum. In some embodiments, the platinum nanoparticles are approximately 3 nm in diameter and are loaded at a concentration of approximately 0.75%. Increasing the concentration of the platinum nanoparticles while maintaining the size of the platinum nanoparticles has been found not to be effective in selectively reducing the olefin functional group over the nitro functional group. Similarly, a smaller platinum size has been found not to be effective in reducing the olefin functional group over the nitro functional group. However, decreasing the concentration while maintaining the size of the platinum has been found to be effective in maintaining the selectivity, but results in decreasing the activity of the reaction.

The reaction illustrated in FIG. 1 is an exemplary reaction to demonstrate selective hydrogenation of the olefin functional group over the nitro functional group by using the advanced catalyst of the present invention, wherein the advanced catalyst contains a specific size and concentration of the catalytic nanoparticles. The olefin functional group of a more complex molecule is able to be reduced using the advanced catalysts of the present invention. For example, synthesis of Taxol®, a potent anti-cancer natural product, is a multi-step process comprising many reactions. The catalyst of the present invention can be used in the multi-step process to achieve the synthesis of Taxol®.

FIGS. 5 illustrates one embodiment of a method 500 of forming and using catalysts in accordance with the principles of the present invention.

At step 510, it is determined what particular catalysis result is desired. In some embodiments, the particular catalysis result is the selective reduction of a selected functional group without the reduction of other functional groups of a molecule (e.g., reducing olefin functional group over nitro functional group, reducing nitro functional group over halide functional group, or reducing ketone functional group over ester functional group). In some embodiments, the particular catalysis result is non-selective hydrogenation, oxidation, isomerization, or carbon-carbon bond formation.

At step 520, a catalyst is formed having catalytic nanoparticles, preferably bonded to support nanoparticles. The catalytic nanoparticles are tuned to a particular size and concentration to achieve the particular catalysis result that is desired. In a preferred embodiment, the catalytic nanoparticles are formed and bonded to the support nanoparticles using a plasma gun. However, it is contemplated that other methods, such as wet chemistry methods, can be employed as well.

At step 530, the catalyst is used in a catalytic process to achieve the particular catalysis result that is desired. In some embodiments, the catalytic process is a batch process. In some embodiments, the catalytic process is a continuous process.

At step 540, a different particular catalysis result is determined. For example, if at step 510, it was determined that the particular catalysis result was to selectively reduce the olefin functional group over the nitro functional group, then at step 540, it might be determined that the particular catalysis result is to selectively reduce the nitro functional group over the halide functional group.

At step 550, similar to step 520, a catalyst is formed. Here, the size and/or concentration of the catalytic material is adjusted to achieve the different particular catalysis result. For example, the size of the catalytic particles might be increased or decreased. Similarly, the concentration of the catalytic material might be increased or decreased. In some embodiments, the size and/or concentration is adjusted, while the chemical elements remain the same. For example, it has been found that a catalyst having nano-sized rhodium-platinum particles bonded to nano-sized aluminum oxide particles is very effective in selective hydrogenation and can be used for different embodiments of selective hydrogenation simply by adjusting the size and/or concentration of the rhodium-platinum particles.

At step 560, similar to step 530, the adjusted catalyst is used in a catalytic process to achieve the particular catalysis result that is different from the previous catalysis result. The adjustment and use of the catalyst can be repeated as many times as desired, with new catalysis results being determined, and catalysts being adjusted and formed to achieve those new catalysis results.

The present invention has been described in terms of specific embodiments incorporating details to facilitate the understanding of principles of construction and operation of the invention. Such reference herein to specific embodiments and details thereof is not intended to limit the scope of the claims appended hereto. It will be readily apparent to one skilled in the art that other various modifications may be made in the embodiment chosen for illustration without departing from the spirit and scope of the invention as defined by the claims.

What is claimed is:

1. A catalyst comprising:
a plurality of support nanoparticles; and
a plurality of catalytic nanoparticles, with at least one catalytic nanoparticle being bonded to each support nanoparticle, the catalytic particles having a size and a concentration, wherein a first configuration of the size and the concentration of the catalytic nanoparticles enables a first catalysis result and a second configuration of the size and the concentration of the catalytic nanoparticles enables a second catalysis result, the first and second configurations having a different size or concentration, and the first and second catalysis results being different.

2. The catalyst of claim 1, wherein:
the first catalysis result is a selective reduction of a first selected functional group without reducing one or more other functional groups of a molecule; and
the second catalysis result is a selective reduction of a second selected functional group without reducing one or more other functional groups of a molecule,
wherein the first and second selected functional groups are different.

3. The catalyst of claim 2, wherein:
the first catalysis result is a selective reduction of the olefin functional group without reducing the nitro functional group of a molecule; and
the second catalysis result is a selective reduction of the nitro functional group without reducing the halide functional group of a molecule.

4. The catalyst of claim 2, wherein:
the first catalysis result is a selective reduction of the olefin functional group without reducing the nitro functional group of a molecule; and
the second catalysis result is a selective reduction of the ketone functional group without reducing the ester functional group of a molecule.

5. The catalyst of claim 2, wherein:
the first catalysis result is a selective reduction of the nitro functional group without reducing the halide functional group of a molecule; and
the second catalysis result is a selective reduction of the ketone functional group without reducing the ester functional group of a molecule.

6. The catalyst of claim 2, wherein the first selected functional group is olefin.

7. The catalyst of claim 2, wherein the first selected functional group is nitro.

8. The catalyst of claim 2, wherein the first selected functional group is ketone.

9. The catalyst of claim 1, wherein the catalytic nanoparticles are platinum nanoparticles.

10. The catalyst of claim 1, wherein the catalytic nanoparticles are alloy nanoparticles.

11. The catalyst of claim 10, wherein the alloy nanoparticles comprise platinum and rhodium.

12. The catalyst of claim 1, wherein the support nanoparticles are aluminum oxide nanoparticles.

13. The catalyst of claim 12, wherein the catalytic nanoparticles are platinum nanoparticles.

14. The catalyst of claim 1, wherein the catalytic nanoparticles are platinum-rhodium nanoparticles.

15. A method of forming a catalyst, the method comprising:
determining a first particular configuration for a first catalyst, wherein the first particular configuration comprises a particular size and concentration of catalytic nanoparticles configured to achieve a first particular catalysis result when the first catalyst is used in a catalytic process; and
forming the first catalyst according to the first particular configuration, wherein the first catalyst comprises a plurality of support nanoparticles each having at least one catalytic nanoparticle bonded to it.

16. The method of claim 15, wherein the first particular catalysis result is a selective reduction of a selected functional group without reducing one or more other functional groups of a molecule.

17. The method of claim 15, wherein the first particular catalysis result is a selective reduction of the olefin functional group without reducing the nitro functional group of a molecule.

18. The method of claim 15, wherein the first particular catalysis result is a selective reduction of the nitro functional group without reducing the halide functional group of a molecule.

19. The method of claim 15, wherein the first particular catalysis result is a selective reduction of the ketone functional group without reducing the ester functional group of a molecule.

20. The method of claim 15, wherein the first selected functional group is olefin.

21. The method of claim 15, wherein the first selected functional group is nitro.

22. The method of claim 15, wherein the first selected functional group is ketone.

23. The method of claim 15, wherein the catalytic nanoparticles are platinum nanoparticles.

24. The method of claim 15, wherein the catalytic nanoparticles are alloy nanoparticles.

25. The method of claim 24, wherein the alloy nanoparticles comprise platinum and rhodium.

26. The method of claim 15, wherein the support nanoparticles are aluminum oxide nanoparticles.

27. The method of claim 26, wherein the catalytic nanoparticles are platinum nanoparticles.

28. The method of claim 26, wherein the catalytic nanoparticles are platinum-rhodium nanoparticles.

29. The method of claim 15, wherein the step of forming the first catalyst comprises:
vaporizing support material and catalytic material using a plasma gun, thereby forming vaporized support material and vaporized catalytic material; and
quenching the vaporized support material and the vaporized catalytic material, thereby forming the support nanoparticles and the catalytic nanoparticles.

30. The method of claim 15, further comprising:
determining a second particular configuration for a second catalyst, wherein the second particular configuration comprises a particular size and concentration of catalytic nanoparticles configured to achieve a second particular catalysis result when the second catalyst is used in a catalytic process; and
forming the second catalyst according to the second particular configuration, wherein the second catalyst comprises a plurality of support nanoparticles each having at least one catalytic nanoparticle bonded to it,
wherein the second catalyst comprises the same chemical elements as the first catalyst, but the second particular configuration differs from the first particular configuration in at least the size or the concentration of catalytic nanoparticles, thereby enabling the second particular catalysis result to be different from the first particular catalysis result.

31. A method of using a catalyst, the method comprising:
providing a finely-tuned catalyst, wherein the finely-tuned catalyst comprises a plurality of support nanoparticles and a plurality of catalytic nanoparticles, with at least one catalytic nanoparticle being bonded to each support nanoparticle; and
using the finely-tuned catalyst in a catalytic process, wherein the finely-tuned catalyst enables selective reduction of a selected functional group without reduction of one or more other functional groups of a molecule.

32. The method of claim 31, wherein the selected functional group is olefin.

33. The method of claim 32, wherein the finely-tuned catalyst enables selective reduction of the olefin functional group without reduction of the nitro functional group.

34. The method of claim 31, wherein the selected functional group is nitro.

35. The method of claim 34, wherein the finely-tuned catalyst enables selective reduction of the nitro functional group without reduction of the halide functional group.

36. The method of claim 31, wherein the selected functional group is ketone.

37. The method of claim 36, wherein the finely-tuned catalyst enables selective reduction of the ketone functional group without reduction of the ester functional group.

38. The method of claim 31, wherein the catalytic nanoparticles are platinum nanoparticles.

39. The method of claim 31, wherein the catalytic nanoparticles are alloy nanoparticles.

40. The method of claim 39, wherein the alloy nanoparticles comprise platinum and rhodium.

41. The method of claim 31, wherein the support nanoparticles are aluminum oxide nanoparticles.

42. The method of claim 41, wherein the catalytic nanoparticles are platinum nanoparticles.

43. The method of claim 41, wherein the catalytic nanoparticles are platinum-rhodium nanoparticles.
